(19) United States
(12) Patent Application Publication
(10) Pub. No.: US 2004/0015121 A1
(43) Pub. Date: Jan. 22, 2004

(54) Container having a fluid transfer tube with textured inner surface and method for making the container

(75) Inventors: Patrick T. Ryan, Crystal Lake, IL (US); Robert R. Roberts, Burlington, WI (US); Randy J. Koppen, McHenry, IL (US); Robert J. Kristan JR., Round Lake, IL (US); Joseph Nolan Veillon JR., McHenry, IL (US); Robert Gettens, Wilmington, IL (US)

Correspondence Address: Jeffrey Nichols, Baxter Healthcare Corporation, One Baxter Parkway, DF3 - 2E, Deerfield, IL, 60015, US

(73) Assignee: Baxter International, Inc.

(21) Appl. No.: 10/160,660

(22) Filed: May 31, 2002

(51) Int. Cl. A61N 1/00

(52) U.S. Cl. 604/19

(57) ABSTRACT

A container for retaining a fluent therapeutic has a fluid transfer tube having a proximal end in fluid communication with an interior space of the container, a distal end accessible outside of the container and an inner surface defining a flow passage through which fluent therapeutic is delivered from the interior space of the container. At least a portion of the tube inner surface is textured such that the inner surface has a surface roughness equal to or greater than about 20 microinches. In a method for making such a container, the fluid transfer tube is extruded from an extruder and its inner surface is textured to have a surface roughness equal to or greater than about 20 microinches. The tube is secured to the container with a proximal end in fluid communication with the interior space of the container and a distal end accessible outside of the container.

### BACKGROUND OF THE INVENTION

This invention relates generally to a container for retaining a fluent therapeutic such as blood, saline, glucose, electrolyte and the like for subsequent administration to a medical patient, and more particularly to such a container having a fluid transfer tube capable of releasably receiving a male connector therein to permit dispensing of the fluent therapeutic from the container.

Containers such as intravenous solution bags typically have one or more fluid transfer tubes or “port” tubes providing outlets through which fluent therapeutic is dispensed from the container for delivery to a patient. Each fluid transfer tube is connected to the solution bag, such as by being bonded or welded thereto, with a proximal end of the tube open to the interior of the bag and a distal end disposed exterior of the bag and sealed by a suitable closure. The tube forms a passageway into the interior of the bag and will generally include a sterile barrier such as a pierceable membrane.

An administration set, which may include various known components such as additional delivery tubes, filters, drip chambers, clamps and the like, is connected to the solution bag by a male connector which typically resembles a tubular needle or “spike”. More particularly, the spike is inserted into the distal end of the fluid transfer tube to pierce the membrane. The spike is open at its outer or piercing end and has a flow passage extending therethrough to provide fluid communication between the administration set and the interior space of the solution bag. The administration set is also in fluid communication with the patient for delivering the contents of the solution bag to the patient.

The fluid transfer tube and spike are typically sized relative to each other to provide an interference fit therebetween, such as by frictional engagement of the spike outer surface with the inner surface of the fluid transfer tube, to form a sterile seal therebetween and to inhibit the spike against unintended withdrawal from the tube. However, one administration set may be used to infuse several containers of fluid into a patient. Therefore, the spike is desirably capable of insertion in and removal from the tube upon application of a relatively low insertion or withdrawal force to the spike, such as by pushing or pulling on the spike. To this end, U.S. Pat. No. 4,162,220 to Servas discloses a blood filter having a blood inlet spike insertable into a tubular outlet of a blood bag. The outer surface of the spike is roughened to facilitate insertion of the spike into the tubular outlet of the blood bag.

In view of different manufacturing tolerances and processes practiced by existing spike manufacturers, it is common for spikes of the same size and intended purpose to vary in outer surface roughness. These small variations in surface roughness can result in substantially different spike removal forces needed to withdraw the spike from the fluid transfer tube of the container.

One common method of producing a fluid transfer tube such as the tube described above for transferring fluent therapeutic from the solution bag is to extrude the tube from an extruder. Conventional extruders include a cylinder, or barrel having a receiving end, an extrusion end and a screw extending from the receiving end to the extrusion end of the barrel and rotatable on its longitudinal axis. Material to be extruded, such as a polymeric material, is loaded into the receiving end of the barrel and the screw is rotated such that the flight of the screw works the material through the barrel toward its extrusion end. The material is melted as it moves through the barrel.

Upon reaching the extrusion end of the barrel, the melted material is pumped through a die having an annular opening shaped and sized according to the desired tube dimensions. A die pin extends coaxially within the die and has a gas passage in fluid communication with a source of gas, such as air, for directing low-pressure air to flow axially (e.g., in the direction of extrusion) within the tube as the tube is extruded. The air flows from the die to maintain the inflated tubular shape of the tube until the material is sufficiently solidified so that the tube will not collapse upon itself.

It is also known in the art to direct pressurized air to impact the outer surface of the tube as the tube is extruded from the die to promote rapid cooling of the tube outer surface. This rapid cooling causes the formation of small fractures in the tube outer surface to give the tube a relatively frosted appearance and to make the tube less tacky and thus easier to handle during subsequent handling operations.

### SUMMARY OF THE INVENTION

In general, a container of one embodiment of the present invention for retaining a fluent therapeutic to be administered to a medical patient comprises an interior space for retaining the therapeutic and a fluid transfer tube having a proximal end in fluid communication with the interior space. A distal end of the fluid transfer tube is accessible outside of the container. An inner surface of the tube defines a flow passage between the proximal and distal ends through which the fluent therapeutic is delivered from the interior space of the container. At least a portion of the tube inner surface is textured such that the inner surface has a surface roughness equal to or greater than about 20 microinches.

A method of the present invention for making a container for retaining a fluent therapeutic to be delivered to a medical patient generally comprises extruding the tube from an extruder whereby the tube has an inner surface defining a flow passage therethrough. The inner surface of the tube is textured to have a surface roughness equal to or greater than about 20 microinches. The tube is secured to the container such that a proximal end of the tube is in fluid communication with the interior space of the tube and a distal end of the tube is accessible outside of the container.

### BRIEF DESCRIPTION OF THE DRAWINGS

FIG. 1 is a perspective of a container of the present invention in the form of a solution bag having fluid transfer tubes secured thereto, and a male connector in the form of a spike to be releasably connected to one of the fluid transfer tubes;

FIG. 2 is a perspective of the fluid transfer tube of FIG. 1 with a portion of the tube fragmented to show a textured inner surface thereof;

FIG. 3 is a schematic of an extruder die and a die pin for use with an extruder in accordance with a method of the present invention for making the fluid transfer tube of the container of FIG. 1;

FIG. 4 is an enlarged schematic of a portion of the die pin of FIG. 3;

FIG. 5 is a schematic of a portion of a second embodiment of a die pin; and

FIG. 6 is a schematic of a portion of a third embodiment of a die pin.

Corresponding parts are designated by corresponding reference characters and numerals throughout the several views of the drawings.

### DESCRIPTION OF THE PREFERRED EMBODIMENT

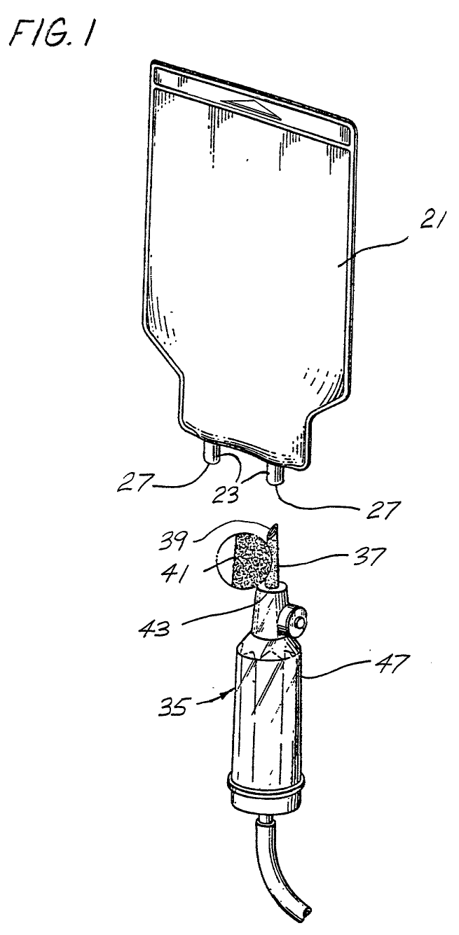

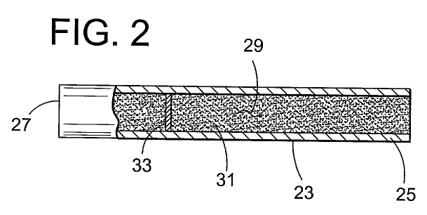

With reference now to the drawings, and in particular to FIG. 1, a container of one embodiment of the present invention is illustrated and described herein with reference to a solution bag 21 of the type commonly used for retaining fluent therapeutics such as blood, saline, glucose, electrolyte or the like to be administered intravenously to a medical patient. The solution bag 21 is constructed of a flexible material, such as by sealing together opposed layers of a polymeric film material generally at the edge margins thereof to define an interior space (not shown) of the bag, and is capable of collapsing as fluid is discharged from the interior space of the bag. Fluid transfer tubes 23 of the present invention are secured to the solution bag 21, such as by being bonded or welded thereto in sealing engagement with the solution bag. Each fluid transfer tube 23 has a proximal (e.g., first) end 25 (not visible in FIG. 1, but shown in FIG. 2) disposed generally within the interior space of the bag 21 to receive fluid into the tube and a distal (e.g., second) end 27 accessible from outside the bag.

An inner surface 31 of the tube 23 defines a flow passage 29 (FIG. 2) extending between the proximal and distal ends 25, 27 of the tube 23. As an example, the fluid transfer tube 23 of the illustrated embodiment has an overall length of about one inch, an inside diameter of about 0.193±0.003 inches and a wall thickness of about 0.032±0.002 inches. The distal end 27 is frequently sealed by a closure (not shown) removably received on the distal end of the tube 23. To provide a sterile barrier, the flow passage 29 may be sealed by a membrane 33 (FIG. 2) disposed within the flow passage, e.g., generally toward the distal end 27 of the tube 23, in sterile sealing engagement with the inner surface 31 of the tube. However, it is understood that the membrane 33 may be omitted without departing from the scope of this invention.

In accordance with one aspect of the present invention, the inner surface 31 of the fluid transfer tube 23 is generally textured (e.g., roughened) to have a desired surface roughness. As used herein, surface roughness refers to the extent and average size of surface irregularities, and more particularly to a measurement of the average deviation of surface irregularities from a hypothetical perfectly smooth surface. Such irregularities may be random or uniformly patterned. The surface roughness may be expressed either as microinches or as micrometers (e.g, microns) and may be measured by any suitable surface roughness measuring device.

For example, one such device (not shown) is a contact-type measuring device available from Mitutoyo America Corporation of Aurora, Ill., U.S.A, under the model designation SV-C500/600 Formtracer, Series 525. This device includes a stylus (not shown) which can be inserted into the flow passage 29 of the tube 23 in contact with the inner surface 31 and moved over the inner surface, such as by rotating the tube relative to the stylus or by rotating the stylus relative to the tube. The deflection of the stylus is measured at intermittent locations along the tube inner surface 31 (e.g., about the inside circumference and/or along the length of the tube) and then averaged by the device to determine the average surface roughness of the tube inner surface. Another contact-type surface roughness measuring device is disclosed in U.S. Pat. No. 6,357,286, the entire disclosure of which is incorporated herein by reference.

It is contemplated that non-contact-type surface roughness measuring devices, wherein surface roughness measurements are taken without contacting the surface being measured, may also be used to determine the average surface roughness of the tube inner surface 31 without departing from the scope of this invention. Examples of such non-contact-type devices are disclosed in U.S. Pat. Nos. 5,757,496; 5,859,919 and 6,163,973, the entire disclosures of which are incorporated herein by reference.

As an example, the inner surface of fluid transfer tubes of conventional containers are generally considered to be smooth and have a surface roughness as measured using the contact-type measuring device described above of about 12 microinches. In contrast, the surface roughness of the tube inner surface 31 of the present invention is preferably equal to or greater than about 20 microinches, more preferably equal to or greater than about 30 microinches, and still more preferably equal to or greater than about 40 microinches.

The fluid transfer tube 23 of the illustrated embodiment is textured over substantially its entire inner surface 31. However, it is contemplated that only a portion of the tube inner surface 31 may be textured, such as by texturing a circumferential band (not shown) or multiple bands (not shown) of the inner surface, or by texturing longitudinally extending portions of the inner surface such to form pin stripe or helical stripe patterns, or by forming generally any pattern, regular or random, in which a portion of the tube inner surface is textured and a portion of the tube inner surface remains substantially non-textured.

The fluid transfer tube 23 of the illustrated embodiment is constructed of an elastic material capable of stretching to receive a male connector 37 within the flow passage 29 in frictional engagement with the tube inner surface 31 so that the tube generally grips and holds the connector within the flow passage. For example, the tube 23 may be constructed of various polymeric materials including materials having PVC or materials which are devoid of PVC. However, it is understood that the tube 23 may instead be constructed of a generally inelastic material without departing from the scope of this invention.

Referring still to FIG. 1, the male connector is shown and described herein as a tubular spike 37 sized for insertion through the distal end 27 of the tube 23 into the flow passage 29. The spike 37 of the illustrated embodiment is generally conical and includes a base 43 and a beveled outer end 39 which forms a sharp tip for piercing the membrane (not shown) sealing the flow passage, if such a membrane is present. However, the spike 37 may be frusto-conical, cylindrical or other suitable shape without departing from the scope of this invention as long as the spike is capable of insertion into the flow passage 29 of the tube 23. The outside diameter of the spike 37 is sized about equal to or slightly larger than the inside diameter of the tube for frictional engagement between an outer surface 41 of the spike and the textured portion of the tube inner surface 31 to secure the spike against unintended withdrawal from the tube 23. As an example, in one embodiment the outside diameter of the spike 37 gradually increases from about 0.198 inches at its outer end 39 to about 0.213 inches at its base 43. The tubular spike 37 is open at its outer end 39 and has a central passage (not shown) therein in fluid communication with its open outer end for receiving fluent therapeutic from the fluid transfer tube 23 into the spike. While the spike 37 of the illustrated embodiment is shown as having a roughened outer surface 41, it is understood that the spike outer surface may be generally smooth without departing from the scope of this invention.

In the illustrated embodiment, the spike 37 forms part of a spike assembly, generally indicated at 35, which includes a spike holder 47. However, it is understood that the spike holder 47 may be omitted without departing from the scope of this invention. An administration set (not shown) is connected to the spike assembly 35 in fluid communication with the central passage of the spike 37 for administering fluent therapeutic received by the spike assembly to a medical patient. Construction and operation of administration sets is well know in the art and will not be further described herein.

In operation, the spike assembly 35 is releasably connected to the solution bag 21 by grasping the spike holder and inserting the spike 37, outer end 39 first, through the distal end 27 of the fluid transfer tube 23 into the flow passage 29 to pierce the tube closure. The spike 37 is pushed further into the flow passage 29 such that the proximal end of the tube generally stretches to accept the spike and then grip the outer surface 41 of the spike such that the spike outer surface is held in frictional engagement with at least a portion of the textured inner surface 31 of the tube. In this position, the open outer end 39 of the spike 37 is in fluid communication with the contents of the interior space of the solution bag 21 via the flow passage 29 through the tube 23 to permit fluent therapeutic from the solution bag to be delivered through the administration set to a patient. To disconnect the spike assembly 35 from the solution bag 21, an axially outward directed force (referred to further herein as a spike removal force) must be applied to the spike 37, such as by pulling on the spike holder 47 or the spike, to disengage the spike from the tube inner surface 31.

For spikes 37 which have a relatively smooth outer surface, it was found that the removal force needed to withdraw the spike from the tube exceeds that which a health care provider would find to be tolerable. As an example, a spike removal force greater than about 20 pounds would be deemed to be excessive by many health care providers. For spikes 37 similar to that shown in FIG. 1 as having a relatively rough outer surface, it was found that the spike removal force varied little relative to the surface roughness of the interior of the tube 23. Thus for spikes 37 having a relatively smooth outer surface the spike removal force is generally inversely proportional to the surface roughness of the tube inner surface 31. That is, as the surface roughness of the tube inner surface 31 increases, the spike removal force needed to withdraw the spike from the tube 23 generally decreases. As an example, the force required to remove the spike 37 from the fluid transfer tube 23 of FIG. 1 is in the range of about 5 and about 20 lbf. (e.g., pounds-force) and more frequently in the range of about 10 to about 15 lbf. However, it is understood that the spike removal force may vary depending on the surface roughness of the tube inner surface 31 and the relative sizes of the tube 23 and spike 37.

### EXAMPLE

A test was conducted to determine 1) the relationship between the force required to withdraw the spike 37 from the tube 23 and the surface roughness of the tube inner surface 31, and 2) the relationship between the force needed to insert the spike into the flow passage 29 of the tube 23 and the surface roughness of the tube inner surface. One set of fluid transfer tubes 23, each having a length of about one inch, an inner diameter of about 0.193 inches and a wall thickness of about 0.032 inches, was constructed such that the tubes had a generally smooth or otherwise non-textured inner surface, e.g., a measured inner surface roughness of less than about 15 microinches and a mean (e.g., average of all tubes measured) surface roughness of about 12.8 microinches.

A second set of tubes having substantially the same dimensions as the first set of tubes was constructed such that each tube had a textured inner surface. The surface roughness of each textured inner surface was measured using the device described previously as being available from Mitutoyo America Corporation of Aurora, Ill. under the model designation SV-C500/600 Formtracer, Series 525. An average surface roughness was then determined and recorded for each textured inner surface. The second set of tubes had a measured inner surface roughness in the range of about 35 to about 43 microinches and a mean (e.g., average of all tubes measured) surface roughness of about 39 to about 40 microinches.

Spikes having a generally textured outer surface 41 similar to the spike 37 shown in FIG. 1 were measured for outer surface roughness using a conventional profilometer. For example, one such profilometer is available from Taylor-Hobson of Leicester, England under the model designation Surtronic 3. The spikes 37 were secured in a standard loading device capable of moving the spikes axially into and subsequently outward from the flow passages 29 of the tubes 23. The device first moved the spikes (e.g., one at a time) at a speed of about 20 inches per minute toward and into the flow passages 29 of the two sets of tubes 23 until the outer surface of each spike frictionally engaged the inner surface of each tube. A force measurement instrument available from Instron under the model designation 5565 was used to monitor and record the force required to insert the spikes 37 into the tubes 23.

The device was then operated to pull the spikes outward from the flow passages 29 of the two sets of tubes 23 at a rate of about 20 inches per minute until the spikes were fully removed from the tubes. The force required to remove the spikes from the tubes (e.g., the spike removal force) was monitored and recorded by the force measurement device described above.

For the first set of tubes, e.g., having the generally non-textured inner surfaces, the force required to insert the spikes into the tubes was in the range of about 9 to about 15 lbf. The mean insertion force for the first set of tubes was about 12.4 lbf. For the second set of tubes, e.g., having inner surfaces textured in accordance with the present invention, the mean spike insertion force was about 12 lbf. and ranged from about 9 lbf. to about 14.5 lbf. Thus, the force required to insert the spikes 37 into the tubes 23 is relatively independent of the surface roughness of the inner surfaces of the tubes.

The mean force required for removing the spikes 37 from the first set of tubes having the generally non-textured inner surfaces was about 22.7 lbf. and ranged from about 10 to about 32 lbf. In contrast, the mean spike removal force for removing the spikes from the second set of tubes having the textured inner surfaces was about 9.5 lbf. and ranged from about 5 to about 14 lbf. The spike removal force thus substantially decreased by texturing the inner surface of the tube.

While the fluid transfer tube 23 is shown and described herein in connection with a solution bag 21 used to contain fluent therapeutic to be delivered to a medical patient, it is understood that the fluid transfer tube may be used for delivering substantially any type of fluid from substantially any fluid source, as long as the tube has a textured inner surface 31 and is capable of receiving a male connector 37 therein in engagement with the textured inner surface of the tube to permit fluid transfer from the tube to the connector. Also, while not shown in the drawings, it is contemplated that the male connector 37 may be disposed upstream of the fluid transfer tube 23, e.g., by being secured to the fluid container 21 or at least located intermediate the fluid container and the fluid transfer tube. In such a configuration, the tube is connected to the male connector by sliding the tube over the outer surface of the connector such that the connector is received in the flow passage 29 of the tube.

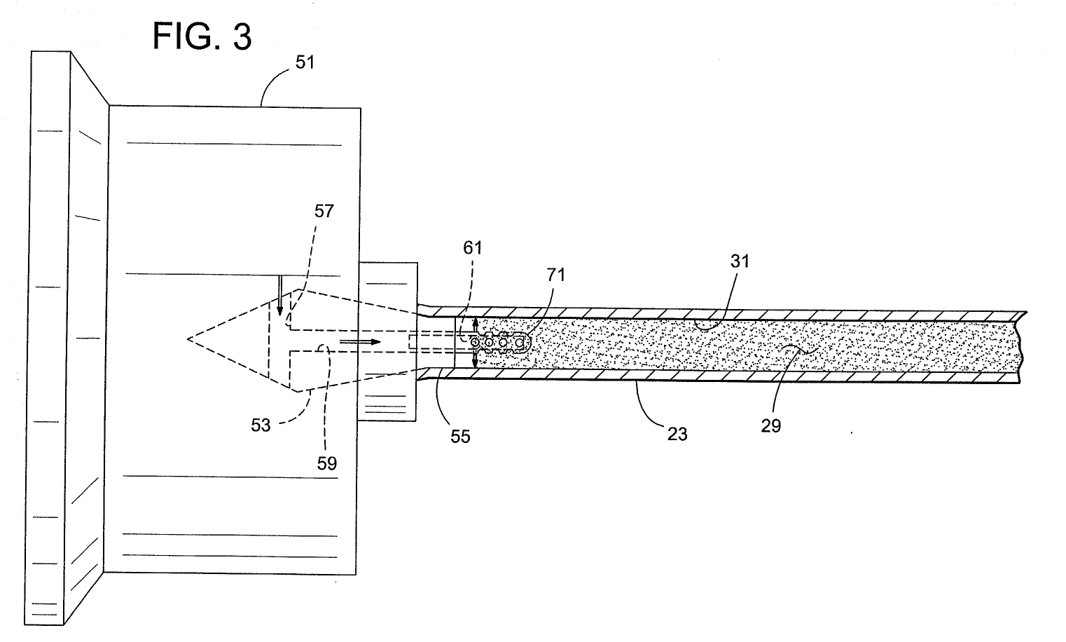

FIG. 3 schematically illustrates a die 51 for an extruder (not shown) used to extrude articles from a polymeric material, and more particularly for extruding a fluid transfer tube 23 of the present invention having a textured inner surface 31. The extruder conventionally comprises a barrel (not shown) having a receiving end, an extrusion end and a screw (not shown) extending from the receiving end to the extrusion end of the barrel and being rotatable on its longitudinal axis. The die 51 is positioned adjacent the extrusion end of the barrel and has an annular opening (not shown) through which material is extruded in the form of a tube 23. For example, the inner diameter and thickness of the annular opening corresponds to the desired cross-sectional dimensions of the extruded tube 23.

A die pin 53 is positioned in the die 51 coaxial with the annular die opening and extends out beyond the opening in the direction of extrusion to an outer end 55 of the die pin. The die pin 53 has a gas inlet 57 in fluid communication with a source (not shown) of pressurized gas, such as compressed air, for receiving gas into the die pin. An axial gas passage 59 the die pin 53 communicates with the gas inlet 57 and extends to an outlet 61 disposed at the outer end 55 of the die pin in coaxial relationship with the annular die opening. Construction of the extruder to the extent set forth above is well known to those skilled in the art and therefor will not be further described herein except to the extent necessary to describe the present invention.

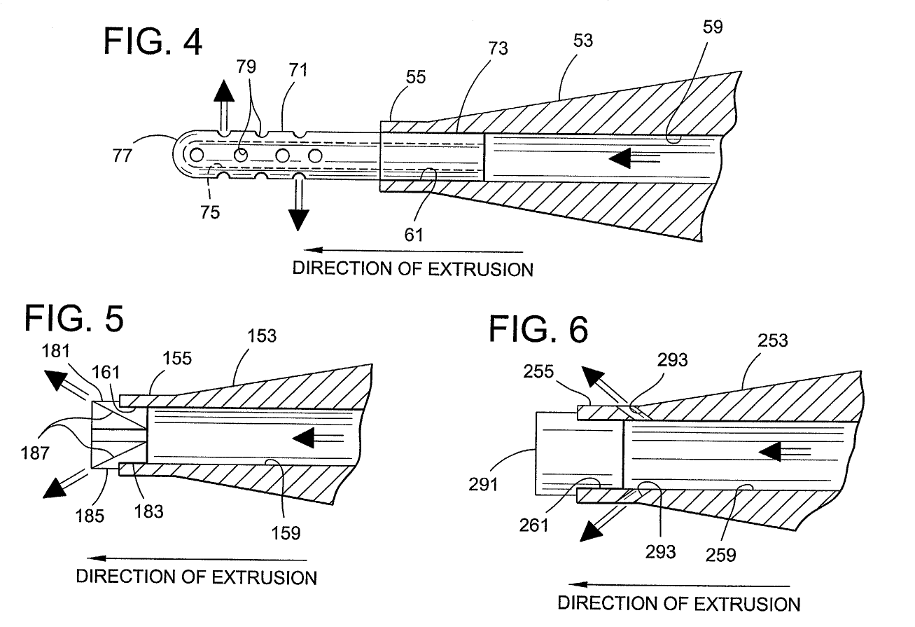

In accordance with one aspect of a method and apparatus of the present invention for making a fluid transfer tube 23 having a textured inner surface 31, the die pin 53 is further configured to exhaust gas from the gas passage 59 generally radially outward (as indicated by the flow arrows in FIG. 3) relative to the to direction of extrusion (e.g., normal thereto) to impact the inner surface of the tube 23 as the tube is extruded from the die 51. For example, as shown in FIGS. 3 and 4, the die pin 53 further comprises a tubular exhaust member 71 secured to the outer end 55 of the die pin 53 in fluid communication with the gas passage 59 and extending axially further beyond the annular die opening in coaxial relationship therewith.

In the illustrated embodiment of FIGS. 3 and 4, the exhaust member is sized at an inner end 73 thereof for a press fit within the gas passage 59 at the outer end 55 of the die pin 51. The exhaust member has an axial passage 75 in fluid communication with the die pin gas passage 59 and a closed outer end 77. Exit holes 79 are formed in the side wall of the exhaust member 71 in longitudinally and circumferentially spaced relationship with each other to exhaust substantially all of the gas from the exhaust member radially outward relative to the direction of extrusion to impact the inner surface 31 of the tube 23 being extruded. It is understood, however, that the outer end 77 of the exhaust member 71 may instead be partially or fully open so that only a portion of the gas is exhausted through the side holes 79 without departing from the scope of this invention.

As an example, the exhaust member 71 shown in FIG. 4 can be used with a conventional extruder to extrude the fluid transfer tube 23 of the container 21 of FIG. 1 and has length of about 1.5 inches. The exit holes 79 are each 1/16 inches in diameter and are spaced longitudinally about 0.25 inches apart along the length of the exhaust member. There are four axial rows of exit holes 79 spaced circumferentially about the exhaust member 77. However, it is understood that the size of the exhaust member 77 may vary, and that the size and number of exit holes 79, as well as the location of the exit holes in the exhaust member, may also vary without departing from the scope of this invention.

In operation, material from which the tube 23 is to be extruded is fed to the receiving end of the barrel. The screw rotates continuously within the barrel such that the flight of the screw works the material through the barrel toward the extrusion end thereof. The material is melted while being moved through the barrel and, upon reaching the extrusion end, the melted material is forced through the annular die opening to form a tube 23. As the tube 23 is extruded from the die 51, gas is directed to flow through the die pin gas passage 59 into the exhaust member 71. Gas is exhausted from the exhaust member 71 through the side holes 79 and flows radially outward relative to the direction of the extrusion to impact the inner surface 31 of the side of the tube 23 as the tube is extruded from the annular opening of the die 51 to promote rapid cooling of the tube inner surface. This rapid cooling causes small fractures to form in the tube inner surface 31 to thereby provide a textured inner surface of the tube 23. The fluid transfer tube 23 is then cut to the desired length and secured to a container as described previously.

FIG. 5 illustrates a second embodiment of a die pin 153 wherein a plug 181 is press fit into the outlet end 155 of the gas passage 159 of the die pin. The plug 181 has a stepped diameter to define an inner portion 183 sized for insertion into the outlet end 155 of the gas passage 155 in sealing engagement therewith and an outer portion 185 having a diameter sized larger than the outlet end 155 of the gas passage 159 to seat against the end surface of the die pin upon insertion of the inner portion of the plug into the gas passage. The plug 181 has slits 187 formed therein generally at an angle relative to the axial direction in which the tube 23 is extruded from the die. The slits 187 direct gas received by the plug to flow outward relative to the direction of extrusion to impact the inner surface 31 of the tube as the tube is extruded from the die.

FIG. 6 illustrates a third embodiment of a die pin 253 similar to that of the second embodiment but with a plug 291 sealingly closing the gas passage 259 at the outer end 255 of the die pin. Exit openings 293 are formed in the die pin 253 generally adjacent its outer end 255 in fluid communication with the gas passage 259 for exhausting gas from the gas passage. The exit openings 293 are angled downstream and radially outward relative to the direction of extrusion such that gas exhausted from the gas passage 259 through the openings impacts the inner surface 31 of the tube 23 as the tube is extruded from the annular opening of the die 51.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A container for retaining a fluent therapeutic to be administered to a medical patient, said container comprising an interior space for retaining said therapeutic and a fluid transfer tube having a proximal end in fluid communication with said interior space, a distal end accessible outside the container and an inner surface defining a flow passage between said proximal and distal ends through which said fluent therapeutic is delivered from the interior space of the container, at least a portion of the tube inner surface being textured such that said inner surface has a surface roughness equal to or greater than about 20 microinches.

2. A container as set forth in claim 1 wherein the inner surface of the tube has a surface roughness of at least about 30 microinches.

3. A container as set forth in claim 1 wherein the inner surface of the tube has a surface roughness of at least about 40 microinches.

4. A container as set forth in claim 1 wherein substantially the entire inner surface of the fluid transfer tube is textured.

5. A container as set forth in claim 1 wherein the tube inner surface has a circumference, said textured portion of the inner surface being substantially continuous about the circumference thereof.

6. A container as set forth in claim 1 wherein the fluid transfer tube is adapted to receive a connector through the distal end thereof into the flow passage for frictional engagement with the textured portion of the tube inner surface to releasably secure the connector within the tube.

7. A container as set forth in claim 6 wherein the frictional engagement between the connector and the texture portion of the tube inner surface is such that a removal force must be applied to the connector to withdraw the connector from the tube, said removal force being less than or equal to about 20 lbf.

8. A container as set forth in claim C7 wherein the removal force is in the range of about 10 lbf. to about 15 lbf.

9. A container as set forth in claim 6 wherein the tube is constructed of an elastic material to permit stretching of the tube to accept the connector into the flow passage and then grip and hold the connector therein in frictional engagement with the textured portion of the inner surface of the tube.

10. A container as set forth in claim 1 in combination with a connector adapted for insertion within the flow passage for frictional engagement between an outer surface of the connector and the textured portion of the tube inner surface to releasably secure the connector in the tube.

11. A container as set forth in claim 10 wherein at least a portion of the outer surface of the connector is textured and adapted for engagement with the textured portion of the tube inner surface.

12. A container as set forth in claim 1 wherein the container is constructed of a flexible material.

13. A container as set forth in claim 12 wherein the container is constructed of a polymeric film material.

14. A container as set forth in claim 12 wherein the container is generally collapsible as fluent therapeutic is dispensed from said container through the fluid transfer tube.

15. A method for making a container for retaining a fluent therapeutic to be delivered to a medical patient, said container comprising an interior space and a fluid transfer tube for delivering said fluent therapeutic out of the interior space of the container, said method comprising:
extruding the tube from an extruder whereby the tube has an inner surface defining a flow passage therethrough;
texturing the inner surface of the tube to have a surface roughness equal to or greater than about 20 microinches; and
securing the tube to the container such that a proximal end of the tube is in fluid communication with the interior space of the tube and a distal end of the tube is accessible outside of the container.

16. A method as set forth in claim 15 wherein the step of texturing the inner surface of the tube is conducted as the tube is extruded from the extruder.

17. A method as set forth in claim 16 wherein the texturing step comprises directing gas to flow toward the inner surface of the tube as the tube is extruded from the extruder.

18. A method as set forth in claim 17 wherein the tube is extruded from the extruder in a direction of extrusion, the step of directing gas toward the tube inner surface comprising directing gas to flow within the flow passage of the tube in a direction generally non-axial to the direction of extrusion of the tube.

19. A method as set forth in claim 18 wherein the gas is directed to flow within the tube in a direction normal to the direction of extrusion.

20. A method as set forth in claim 15 wherein the texturing step comprises texturing the inner surface of the tube to have a surface roughness of at least about 30 microinches.

21. A method as set forth in claim 15 wherein the texturing step comprises texturing the inner surface of the tube to have a surface roughness of at least about 40 microinches.
